(19) United States
(12) Patent Application Publication
(10) Pub. No.: US 2010/0145288 A1
(43) Pub. Date: Jun. 10, 2010

(54) Anti-Spill Urinal

(76) Inventor: Joseph L. Mathews, J.L., Rosville, OH (US)

Correspondence Address: MILLER & BARNS, PLLC, 230 EAST BROADWAY, SUITE 250, GRANVILLE, OH, 43023, US

(21) Appl. No.: 12/330,931

(22) Filed: Dec. 9, 2008

(51) Int. Cl. A61M 1/00; A61M 31/00

(52) U.S. Cl. 604/317; 604/93.01

(57) ABSTRACT

The present invention relates to urinals for use by the bedridden. The anti-spill urinal of the present invention includes a flexible tube for improved contact with the patient. The present invention may also include a rounded lip for improved contact with the patient. The urinal of the present invention may include a substantially closed vessel having a bottom wall, side walls, and a top wall with an opening in the top wall of the vessel in combination with a tube in fluid connection with the opening. Preferably, the tube has a number of flexible sections, separated by a number of rigid sections to increase patient comfort and reduce spills. The present invention may also include an extensible section at the distal end of the tube and a graduated section formed in a sidewall of the vessel for measuring the volume of fluid within the vessel.

### TECHNICAL FIELD AND INDUSTRIAL APPLICABILITY OF THE INVENTION

The present invention relates to portable urine collection devices, typically referred to as urinals.

### BACKGROUND OF THE INVENTION

Hospital patients and residents of nursing home with difficulty moving from a bed are often fitted with a Foley catheter. The Foley catheter is a thin, sterile tube inserted into the bladder to drain urine. The insertion of the Foley catheter may be uncomfortable for the patient and may lead to an increase risk of urinary tract infections. The Foley catheter is used because there are no other clean and leak-proof devices for collecting urine.

Currently available urinals rely on a large opening with a cap to seal the urinal after use. One example is U.S. Pat. No. 6,119,280 to Rentsch, issued Sep. 19, 2000 and entitled “All Plastic Leak-Proof Urinal.” The '280 patent recites: The cap has a flat disk-like diaphragm with a reinforced area in the middle thereof. When the cap is placed over the neck and the reinforced area is pushed, the lid takes on a somewhat conical shape which causes it to slide through the neck to a location where the bead confronts the depression. The reinforced area is released and the memory of the plastic of the lid forces the bead into the depression. A handle on the bottle is spaced from the bottle to form a space which has a cove area that locks onto a rail on a hospital bed.

Another example is U.S. Pat. No. 5,778,461 to Attaway, issued Jul. 14, 1998 and entitled “Bedpan with Integral Urinal.” The '461 patent includes a flexible tube that is extendible and has an enlarged funnel-shaped inlet end that functions as a urinal for male patients.

Urinals are typically manufactured by a blow molding process where a molten tube of plastic held between two halves of a mold and air is injected to conform the molten to the interior walls of the tube. The formed urinal is cooled and removed from the mold.

An example of a blow molded article is shown in U.S. Pat. No. 5,762,221 to Tobias et al., and entitled “Hot-Fillable, Blow-Molded Plastic Container Having A Reinforced Dome” incorporated herein by reference. The '221 patent shows a method of forming a domed container having a threaded section for connection with a cap. The threaded section is formed on the finish of the bottle. The finish being the section of a blow molded article through which compressed gasses are blown to form the article.

The manufacture of thermoplastic containers by blow molding is also disclosed in U.S. Pat. No. 6,213,756 to Czesak et al., and entitled “Extrusion Blow Molding Apparatus For Manufacture Of Thermoplastic Containers” incorporated herein by reference. The '756 patent discloses a threaded or otherwise configured closure receiving neck portion of the container, which is commonly referred to as the “finish” of the container. The blowing of the container is done by air or another gas that is introduced into the parison within the mold cavity by a blow pin that extends through the neck mold into the parison within the body mold.

The manufacture of flexible tubes is disclosed in U.S. Pat. No. 4,216,801 to Aykanian and entitled “Flexible Tube” incorporated herein by reference. The '801 patent discloses the manufacture of flexible tubes, suitable for use as drinking straws, having a thermoplastic tube with a flexible zone which may be flexed without substantial springback. The flexible zone comprises a series of circumferential grooves having an annular floor and two opposed groove sides. The grooves are formed by a process disclosed in U.S. Pat. No. 3,493,998 to Meuller et al. and entitled “Machine For Forming A Flexible Area In Tubular Plastic” incorporated herein by reference.

### SUMMARY OF THE INVENTION

The present invention relates to urinals for use by the bedridden. The anti-spill urinal of the present invention includes a flexible tube for improved contact with the patient. The present invention may also include an extensible tube for improved contact with the patient. The urinal of the present invention may include a substantially closed vessel having a bottom wall, side walls, and a top wall with an opening in the top wall of the vessel in combination with a tube in fluid connection with the opening. Preferably, the tube has a number of flexible sections, separated by a number of rigid sections to increase patient comfort and reduce spills. The present invention may also include a rounded lip section at the distal end of the tube and a graduated section formed in one or more sidewalls of the vessel for measuring the volume of fluid within the vessel. The present invention also provides a method of manufacturing the anti-spill urinal by blow molding a substantially closed vessel having a bottom wall, side walls, and a top wall while maintaining an opening in at least one of these walls, forming a tube with one or more flexible sections, and optionally with a rigid section between two flexible sections.

### BRIEF DESCRIPTION OF THE DRAWING

FIG. 1 is a side, plan view of the improved urinal of the present invention and showing a threaded connector between the vessel and the flexible tube.

FIG. 2 is a top, plan view of the improved urinal of the present invention and showing a friction-fit connector between the vessel and the flexible tube.

### DETAILED DESCRIPTION AND PREFERRED EMBODIMENTS OF THE INVENTION

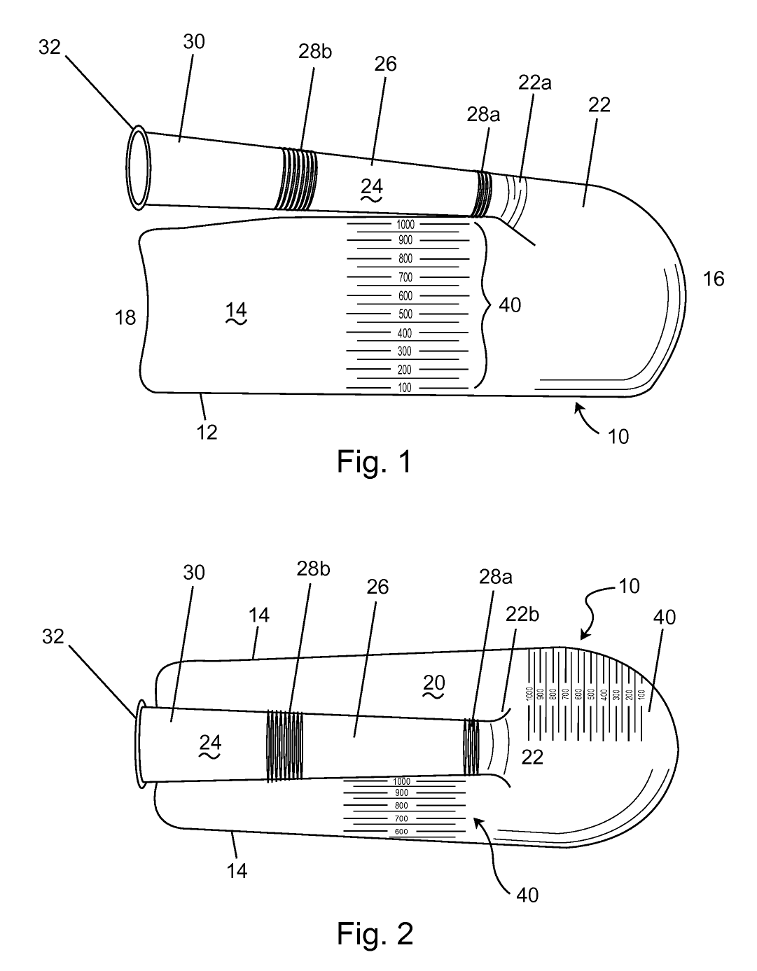

FIG. 1 and FIG. 2 illustrate a blow-molded plastic urinal having vessel 10 formed of a base 12, and sidewalls 14, 16 and 18 and a top wall 20. The sidewalls may be curved to conform to the inner thighs of the patient to improve comfort. The vessel 10 may also be tapered, that is wall 16 may be longer than wall 18 of the vessel to have a generally trapezoidal shape, again to allow the vessel 10 to fit between the patient's thighs.

The vessel 10 includes a finish 22 formed into one or more of the sidewalls 14, 16 or 18 and the top wall 20. The finish 22 is suited to receiving a flexible tube 24 and may include a series of threads 22a (shown in FIG. 1), for a threaded connection to the tube 24, or a relatively flat surface 22b (shown in FIG. 2), for a friction-fit connection to the tube 24. The vessel 10 may optionally include graduation marks 40 on a sidewall of the vessel and may include a second set of graduation marks at an angle orthogonal to marks 40 so that the volume of fluids may be read conveniently in more than one.

The basic blow molding process used to form the vessel 10 of the present invention is well known and is described U.S. Pat. No. 6,213,756. In manufacturing the urinal of the present invention the vessel 10 is formed by a blow molding process and preferably includes base 12, sidewalls 14, 16 and 18, top wall 20, finish 22 and connection 22a or 22b integrally formed with the finish. The vessel 10 is removed from the mold, allowed to cool. The vessel is then connected to tube 24 in a subsequent operation.

As shown in FIG. 1 and FIG. 2, a flexible tube 24 suitable for use with the present invention preferably includes a first flexible portion 28a and a second flexible portion 28b. The tube 24 also includes rigid section 26, disposed between flexible portions 28a and 28b and a second rigid section 30 on the distal end of the tube. Optionally, a rounded lip 32 may be formed at the end distal of tube 24. The tube 24 may be formed by any suitable process, for example blow molding as disclosed in U.S. Pat. No. 6,213,756 and other conventional processes, as disclosed in U.S. Pat. No. 4,216, and U.S. Pat. No. 3,493,998. The tube 24 may be attached to the vessel 10 by and suitable mechanism including a threaded section 22a, a friction fit section 22b or by the use of an adhesive (not shown) or a combination of a threaded section and adhesive or a friction fit section and adhesive.

The invention of this application has been described above both generically and with regard to specific embodiments. Although the invention has been set forth in what is believed to be the preferred embodiments, a wide variety of alternatives known to those of skill in the art can be selected within the generic disclosure. The invention is not otherwise limited, except for the recitation of the claims set forth below.

I claim:

1. A disposable urinal for collecting liquids, comprising:
a substantially closed vessel having a bottom wall, side walls, and a top wall;
an opening in at least one of said walls; and
a tube in fluid connection with said opening and attached to said vessel, the tube having a first flexible section.

2. The disposable urinal of claim 1, wherein the tube further comprises:
at least a second flexible section; and
a first rigid section between the first and second flexible sections.

3. The disposable urinal of claim 2, wherein the tube further comprises a second rigid section on the distal end of the tube.

4. The disposable urinal of claim 3, wherein the tube further comprises a rounded lip section on the distal end of the tube.

5. The disposable urinal of claim 1, wherein the tube further a rounded lip section on the distal end of the tube.

6. The disposable urinal of claim 1, wherein the vessel further comprises:
a graduated section formed in a sidewall of the vessel for measuring the volume of fluid within the vessel.

7. The disposable urinal of claim 1, wherein the vessel has a generally trapezoidal cross section.

8. The disposable urinal of claim 1, wherein the vessel further comprises:
a first graduated section in a sidewall of the vessel for measuring the volume of fluid within the vessel in a first orientation.
a second graduated section in a sidewall of the vessel for measuring the volume of fluid within the vessel in a second orientation.

9. A disposable urinal for collecting liquids, comprising:
a substantially closed vessel having a bottom wall, side walls, and a top wall;
an opening in the top wall of the vessel;
a tube connected to the vessel at a proximal end of the tube and in fluid connection with said opening, the tube having first and second flexible sections, a first rigid section between the first and second flexible sections and a second rigid section at a distal end of the tube.

10. The disposable urinal of claim 9, wherein the tube further comprises a rounded lip section on the distal end of the tube.

11. The disposable urinal of claim 9, wherein the vessel further comprises:
a first graduated section formed in a sidewall of the vessel for measuring the volume of fluid within the vessel.

12. The disposable urinal of claim 11, wherein the vessel further comprises:
a second graduated section formed in a sidewall of the vessel and orthogonal to the first graduated section.

13. A method of forming disposable urinal for collecting liquids, comprising the steps of:
blow molding a substantially closed vessel having a bottom wall, side walls, and a top wall and having an opening in at least one of said walls formed at the finish of the vessel;
forming a tube having a distal end and a proximal end, first and second flexible sections, a first rigid section between the first and second flexible sections and a second rigid section at a distal end of the tube; and
connecting the proximal end of the tube to the finish of the vessel.

14. The method of forming disposable urinal of claim 13, further comprising the step of:
forming a rounded lip on the distal end of the tube.

15. The method of forming disposable urinal of claim 13 the step of blow molding the vessel further comprises the step of:
forming a graduated section in a sidewall of the vessel.

16. The method of forming disposable urinal of claim 15, wherein the step of blow molding the vessel further comprises the step of:
forming a second graduated section in a second wall of the vessel.

17. The method of forming disposable urinal of claim 15, wherein the step of forming the tube includes a blow molding step.

18. The method of forming disposable urinal of claim 15, wherein the step of forming the tube includes an extrusion step.
